(19) United States
(12) Patent Application Publication
(10) Pub. No.: US 2022/0370311 A1
(43) Pub. Date: Nov. 24, 2022

(54) APPLICATION OF HYDROGEN ION WATER AND A MASK CONTAINING HYDROGEN ION WATER

(71) Applicant: Hangzhou Shanshangshui Technology Co., Ltd, Hangzhou City (CN)

(72) Inventors: Yixin Zhu, Hangzhou (CN); Tingting Lu, Hangzhou (CN)

(21) Appl. No.: 17/751,815

(22) Filed: May 24, 2022

(30) Foreign Application Priority Data
May 24, 2021 (CN) 202110562973.4

(51) Int. Cl. A61K 8/19; A61K 8/02; A61K 8/34; A61Q 19/08

(52) U.S. Cl. CPC A61K 8/19; A61K 8/0212; A61K 8/345; A61Q 19/08; A61K 2800/87; A61K 2800/83

(57) ABSTRACT

Hydrogen ion water for use in cosmetic masks and skin care, as well as a method of making the same. The molecular expressions of hydrogen ions in the hydrogen ion water are H⁺(H₂O)₂, H⁺(H₂O)₃, H⁺(H₂O)₄, H⁺(H₂O)₅, and/or H⁺(H₂O)₆, where O is the oxygen atom in a water molecule, H is the hydrogen atom in water molecule, and H⁺ is a positively charged hydrogen ion. A water mask containing hydrogen ions includes the following components in the indicated weight ratios: 0.01-1% sodium chloride; 0.01-20% glycerol; 0.01-20% 1, 3-butanediol; with the rest being hydrogen ionized water.

### CROSS-REFERENCE TO RELATED APPLICATIONS

This Application Claims priority from Chinese Patent Application Number 202110562973.4, field May 24, 2021, the contents and teachings of which are incorporated herein by reference in their entirety.

### BACKGROUND

### Field of Invention

This invention relates to the technical field of cosmetics, in particular to a hydrogen ion water mask for skin care and beauty.

### Description of Related Art

At the root of many kinds of skin problems lies skin aging. The appearance on original intact skin of acne, enlarged pores, dullness, sensitivity, fine lines, and other problems is a sign that the aging process of the skin has begun. The root of skin aging lies in the accumulation of toxic free radicals, which slow down cell metabolism. Also, the speed of cell regeneration cannot keep up with the speed of cell decline. Cells can even become cancerous in some cases.

Many people use facial water masks to protect and rejuvenate skin. Some facial water masks include hydrogen, which can have a powerful antioxidant effect. Traditional facial masks contain a lot of nutrients, which can easily breed bacteria. In order to protect a mask from deterioration, preservatives are added to the mask to prevent the growth of microorganisms, protect products, prolong the shelf life of products, ensure the safety of products, and prevent consumers from possible infection caused by using products contaminated by microorganisms.

### SUMMARY

Unfortunately, conventional facial masks involve deficiencies. For example, skin health requires a microbial balance but, due to the use of preservatives, a conventional mask may destroy the balance of skin microorganisms and the natural ecological environment of skin cells. This results in the mask failing to achieve the objective of long-term skin protection, and possibly even causing side effects. In addition, existing hydrogen-rich water masks can fail to retain enough hydrogen to be effective. The molecular weight of hydrogen is only 2 Dalton. Once a package of the mask is opened, hydrogen will immediately volatilize in the atmosphere, such that the mask no longer contains hydrogen and cannot convey hydrogen to skin cells. What is needed is a facial mask that does not rely on preservatives and that retains hydrogen so it can easily be absorbed into the skin.

To address this need at least in part, an improved technique provides a hydrogen ion water mask that includes hydrogen ions in the form of H⁺(H₂O)₂, H⁺(H₂O)₃, H⁺(H₂O)₄, H⁺(H₂O)₅, and/or H⁺(H₂O)₆. The hydrogen ions differ from the free hydrogen ions in that they are “surrounded” by a plurality of water molecules (e.g., between 2 and 6 water molecules to a hydrogen ion), so that the hydrogen ions are in a stable state and form tiny water molecule groups, which can be easily absorbed into the human body by the skin. Advantageously, a hydrogen ion water mask having these characteristics can retain hydrogen for a long time and can be easily absorbed by the skin.

Certain embodiments are directed to a water-mask material that includes hydrogen ion water in the form of H⁺(H₂O)₂, H⁺(H₂O)₃, H⁺(H₂O)₄, H⁺(H₂O)₅, and/or H⁺(H₂O)₆. In this arrangement, O is oxygen, H is hydrogen, and H⁺ is a positively charged hydrogen ion. The hydrogen ion of the hydrogen ion water is thereby surrounded by between 2 and 6 water molecules.

Other embodiments are directed to a method of making a water mask. The method includes placing pure water in an electric field to form hydrogen ion water in the form of H⁺(H₂O)₂, H⁺(H₂O)₃, H⁺(H₂O)₄, H⁺(H₂O)₅, and/or H⁺(H₂O)₆, wherein O is oxygen, H is hydrogen, and H⁺ is a positively charged hydrogen ion. The hydrogen ion of the hydrogen ion water is thereby surrounded by between 2 and 6 water molecules.

The foregoing summary is presented for illustrative purposes to assist the reader in readily grasping example features presented herein; however, this summary is not intended to set forth required elements or to limit embodiments hereof in any way. One should appreciate that the above-described features can be combined in any manner that makes technological sense, and that all such combinations are intended to be disclosed herein, regardless of whether such combinations are identified explicitly or not.

### BRIEF DESCRIPTION OF THE SEVERAL VIEWS OF THE DRAWINGS

The foregoing and other features and advantages will be apparent from the following description of particular embodiments, as illustrated in the accompanying drawings, in which like reference characters refer to the same or similar parts throughout the different views. The drawings are not necessarily to scale, emphasis instead being placed upon illustrating the principles of various embodiments.

FIGS. 1 and 2 are schematic diagrams of the state and structure of hydrogen ions in the present invention.

FIG. 3 is a flowchart showing an example method of making a water mask.

### DETAILED DESCRIPTION

Embodiments of the improved technique will now be described. One should appreciate that such embodiments are provided by way of example to illustrate certain features and principles but are not intended to be limiting.

An improved technique provides a hydrogen ion water mask that includes hydrogen ions in the form of H⁺(H₂O)₂, H⁺(H₂O)₃, H⁺(H₂O)₄, H⁺(H₂O)₅, and/or H⁺(H₂O)₆. The hydrogen ions are “surrounded” by a plurality of water molecules (e.g., between 2 and 6 water molecules to each hydrogen ion), so that the hydrogen ions are in a stable state and form tiny water molecule groups, which can be easily absorbed into the human body by the skin. Advantageously, a hydrogen ion water mask having these characteristics can retain hydrogen for a long time and can be easily absorbed by the skin.

In some examples, the hydrogen ion concentration in the above-mentioned hydrogen ion water mask is 10¹⁸ to 10²¹ hydrogen ions/liter. According to the demand of human skin for hydrogen ions, the concentration of hydrogen ions can be controlled within a certain range.

In some examples, a water mask containing hydrogen ions includes the following components in the indicated weight ratios: 0.01-1% sodium chloride; 0.01-20% glycerol; 0.01-20% 1, 3-butanediol; where the rest is hydrogen ionized water.

The term “hydrogen ion water” refers to pure water containing hydrogen ions from 10⁻² mol/L to 10⁻⁶ mol/L. The hydrogen in the hydrogen ion water disclosed herein does not exist substantially in the form of hydrogen gas, but in the form of hydrogen ions; otherwise, it is not conducive to the preservation of hydrogen.

Preferably, the above-mentioned water mask containing hydrogen ions contains the following components in weight ratios: 0.5-1% sodium chloride; 5-20% glycerol; 5-20% 1, 3-butanediol; where the rest is hydrogen ionized water.

In this application, pure water is placed in an extremely high electric field to produce small molecular clusters of water and hydrogen ions. Under the action of an extremely high electric field on hydrogen ions and molecular cluster water, the hydrogen bonds of small molecular clusters in water are broken. Hydrogen ions and oxygen atoms after the hydrogen bonds break form electrostatic adsorption, and form multi-hydrated hydrogen ion charged small molecular cluster water, which has a molecular expression of H⁺(H₂O)₂, H⁺(H₂O)₃, H⁺(H₂O)₄, H⁺(H₂O)₅, and/or H⁺(H₂O)₆.

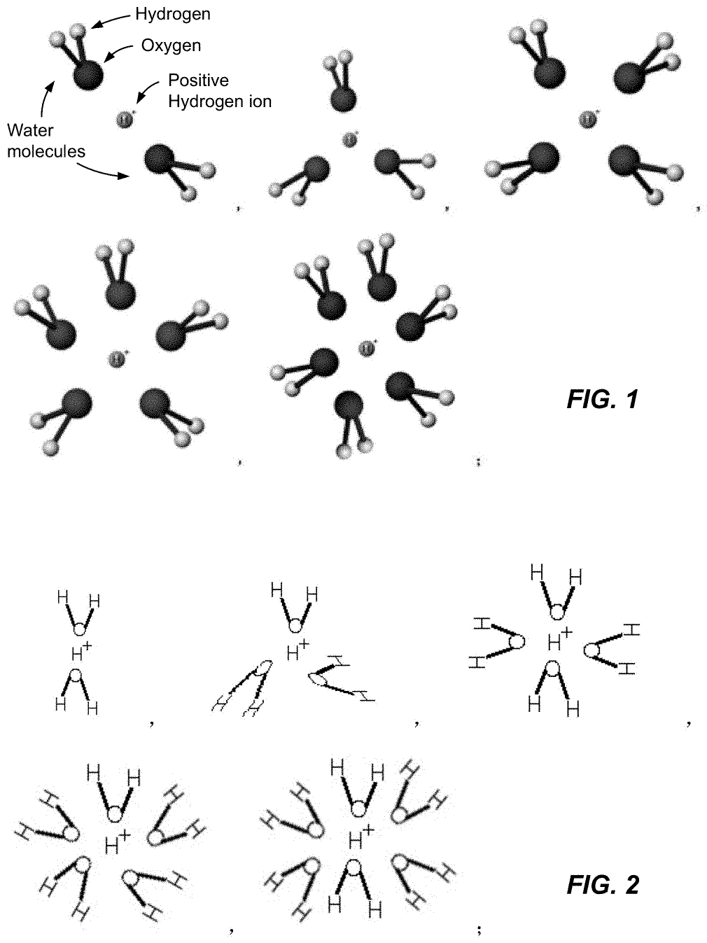

FIGS. 1 and 2 show examples of the resulting molecular expression. In each of these figures, five configurations are shown, each of which includes a positive hydrogen ion (proton) surrounded by two, three, four, five, or six water molecules. Owing to the relative electronegativity of the oxygen atom in each water molecule, the water molecules tend to gather around the positive hydrogen ion with their oxygen atoms facing toward the hydrogen ion. Because hydrogen ions are surrounded by water molecules and water molecules are highly insulated molecules, hydrogen ions can be neutralized by other negative ions or electrons. Its hydrogen ion concentration is 6×10¹⁸˜10²¹ hydrogen ions/liter. Hydrated hydrogen ion charged small molecule cluster water has strong penetrability, which can enter cells smoothly and bring hydrogen ions into cells at the same time.

Hydrogen ion is the basic substance to eliminate oxidative free radicals in organisms, which can selectively neutralize toxic oxidative free radicals, thus delaying skin aging, removing stains, expelling toxin and caring skin.

Hydrogen ion is a safe antioxidant substance, which produces anti-inflammatory reaction at the same time in the antioxidant process. Hydrogen ion is the basic energy for the normal operation of mitochondria (motor) of cells, and has the function of metabolic repair of cells, thus producing anti-apoptosis and cell necrosis without any adverse effects.

In an example, a production process for hydrogen ion water kills all bacteria, producing absolutely sterile liquid. After the mask is packaged, it is sterilized by radiation, thereby producing a sterile and preservative-free hydrogen ion water mask. When being used, the balanced state of natural microorganisms on the skin surface is completely ensured, so that long-term use will not produce any side effects or drug resistance. Use of the product is believed to delay skin aging, remove color spots, detoxify and beautify skin for a long time.

All other antioxidants will inevitably produce by-products and even toxic by-products in the antioxidant process. Hydrogen ion water has the characteristics of small molecule cluster water, which has great diffusion ability and strong penetration. It can enter any area of the body and cells and play an antioxidant effect. Its by-product is water without any toxicity. It can delay skin aging, resist oxidation, inflammation, wrinkle and beauty.

The hydrogen ion water mask has the advantages of high hydrogen ion content, long storage time and good anti-inflammatory, anti-radiation and anti-ultraviolet effects. Effectively repair skin cells damaged by radiation and ultraviolet rays, promote collagen synthesis of fibroblasts, eliminate free radicals and inhibit keratinocyte death, ensure the ecological balance of microorganisms on the skin surface, and achieve the effects of beautifying, eliminating acne, resisting wrinkles and delaying aging.

Hydrated hydrogen ion charged small molecular water acts on skin cells, which can not only repair cell damage, but also stimulate cell regeneration, accelerate cell metabolism, and address skin aging at the root. The complexation of hydrogen ions and glycerol can also balance the secretion of skin hormones, keep a balance between skin water and oil, and inhibit the production of melanin.

Hydrogen hydrate ion charged small molecular group water sub-nanometer size, can penetrate deeply into the basal layer of skin, repair the cells of muscle floor, promote the generation of collagen, and make the skin rejuvenate from the inside out.

Hydrated hydrogen ion charged small molecule water strengthens the skin barrier outside, repairs the muscle bottom inside, and adjusts the skin's “ecological balance,” which is a super skin care effect beyond the reach of many skin care products that “treat the symptoms but not the root causes.”

What is more unique is that the hydrated hydrogen ion charged small molecule water mask does not require preservatives. Eliminating preservatives helps to ensure that the skin flora keeps the original ecological balance and ensures that no allergy or addiction occur.

Small molecular water is composed of 2-6 water molecules, which has strong permeability and strong solubility, and is easy to be absorbed and utilized by every cell in the body, improving the transmission and supply of nutrients in the whole body, bringing in nutrients and bringing out metabolites and toxins. The oxygen content is high (generally more than 6 mg/L, as compared with about 3 mg/L in ordinary water), which can provide more oxygen to tissues and cells in human body, improve the whole-body oxygen transport capacity, enhance the information transmission between each cell and surrounding organizations, and promote the transmission of normal genetic information. As recognized by more and more people, small molecules of water and hydrogen ions is a very effective way to preserve hydrogen in beauty products.

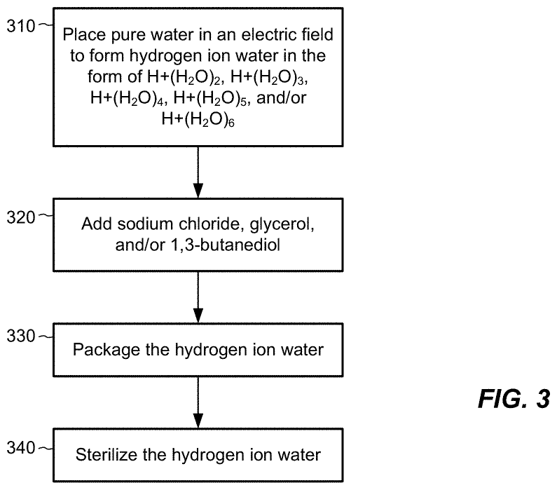

FIG. 3 shows an example method of making a water mask, such as the one described above. As shown, the method includes a step 310 of placing pure water in an electric field to form hydrogen ion water in the form of H+(H₂O)₂, H+(H₂O)₃, H+(H₂O)₄, H+(H₂O)₅, and/or H+(H₂O)₆. At 320, the method further includes adding sodium chloride, glycerol, and/or 1,3-butanediol, e.g., in any of the percentages referred to above. At step 330, the hydrogen ion water is packaged, and at step 340, the packaged material is sterilized, e.g., by exposing it to radiation. The result is a sterile, shelf-stable product that retains a high hydrogen concentration and requires no preservatives.

Various example formulations and their stability over time will now be described.

### Examples 1-5

The following samples were prepared and tested:
Sample 1: Calculated by mass ratio, 0.5% sodium chloride, 10% glycerol, 10% 1, 3-butanediol, the rest is hydrogen ion water, and the concentration of hydrogen ion is 10-3.50 (mol/L);
Sample 2: According to the mass ratio, 0.7% sodium chloride, 5% glycerol, 10% 1, 3-butanediol, the rest is hydrogen ion water, and the concentration of hydrogen ion is 10-4.00 (mol/L);
Sample 3: According to the mass ratio, 1.0% sodium chloride, 20% glycerol, 5% 1, 3-butanediol, the rest is hydrogen ion water, and the concentration of hydrogen ion is 10-4.50 (mol/L);
Sample 4: According to the mass ratio, 0.8% sodium chloride, 6% glycerol, 7% 1, 3-butanediol, the rest is hydrogen ion water, and the concentration of hydrogen ion is 10-5.00 (mol/L).

Test results are summarized in Table 1 below:

TABLE 1

| | Measuring time | | | | | |
|---|---|---|---|---|---|---|
| Item | 0 days | 1 day | 2 days | 3 days | 4 days | 5 days |
| Hydrogen ion | 10^{-3.50} | 10^{-3.50} | 10^{-3.52} | 10^{-3.52} | 10^{-3.52} | 10^{-3.52} |
| concentration | | | | | | |
| in Sample 1 | | | | | | |
| (mol/L) | | | | | | |
| Hydrogen ion | 10^{-4.00} | 10^{-4.02} | 10^{-4.04} | 10^{-4.04} | 10^{-4.04} | 10^{-4.06} |
| concentration | | | | | | |
| in Sample 2 | | | | | | |
| (mol/L) | | | | | | |
| Hydrogen ion | 10^{-4.50} | 10^{-4.50} | 10^{-4.50} | 10^{-4.50} | 10^{-4.52} | 10^{-4.52} |
| concentration | | | | | | |
| in Sample 3 | | | | | | |
| (mol/L) | | | | | | |
| Hydrogen ion | 10^{-5.00} | 10^{-5.00} | 10^{-5.02} | 10^{-5.02} | 10^{-5.02} | 10^{-5.04} |
| concentration | | | | | | |
| in Sample 4 | | | | | | |
| (mol/L) | | | | | | |

Table 2 below shows results for Sample 1 over a longer time scale:

TABLE 2

| | Measuring time | | | | |
|---|---|---|---|---|---|
| Item | 1 week | 2 weeks | 1 month | 2 months | 1 year |
| Hydrogen ion | 10^{-3.54} | 10^{-3.55} | 10^{-3.58} | 10^{-3.62} | 10^{-3.66} |
| concentration in | | | | | |
| Sample 1 (mol/L) | | | | | |

Through the above comparison, it can be found that the mask in the application has a very good preservation effect on ions, and still retains high-depth hydrogen ions during long-term placement. Existing medicine has proved that these small molecular clusters formed by hydrogen ions are easy to be absorbed by human body, which is beneficial to skin and health.

### Example 6

Put a nail in a 500 ml ordinary water bottle, tighten the bottle cap, and after 1 day without any treatment, the whole bottle of water will have rusty color.

### Example 7

A 500 ml bottle is filled with water containing hydrogen ions with a certain concentration in this application, and the concentration of hydrogen ions in the water is 10-3.50 (mol/L). Then a nail is placed in the bottle, and the bottle cap is tightened. After one day without any treatment, the color of the water in the bottle does not change obviously.

Comparing Example 6 with Example 7, it can be seen that the water containing hydrogen ions in this application has obvious antioxidant effect.

Having described certain embodiments, numerous alternative embodiments or variations can be made. Further, although features have been shown and described with reference to particular embodiments hereof, such features may be included and hereby are included in any of the disclosed embodiments and their variants. Thus, it is understood that features disclosed in connection with any embodiment are included in any other embodiment.

As used throughout this document, the words “comprising,” “including,” “containing,” and “having” are intended to set forth certain items, steps, elements, or aspects of something in an open-ended fashion. Also, as used herein and unless a specific statement is made to the contrary, the word “set” means one or more of something. This is the case regardless of whether the phrase “set of” is followed by a singular or plural object and regardless of whether it is conjugated with a singular or plural verb. Also, a “set of” elements can describe fewer than all elements present. Thus, there may be additional elements of the same kind that are not part of the set. Further, ordinal expressions, such as “first,” “second,” “third,” and so on, may be used as adjectives herein for identification purposes. Unless specifically indicated, these ordinal expressions are not intended to imply any ordering or sequence. Thus, for example, a “second” event may take place before or after a “first event,” or even if no first event ever occurs. In addition, an identification herein of a particular element, feature, or act as being a “first” such element, feature, or act should not be construed as requiring that there must also be a “second” or other such element, feature or act. Rather, the “first” item may be the only one. Also, and unless specifically stated to the contrary, “based on” is intended to be nonexclusive. Thus, “based on” should be interpreted as meaning “based at least in part on” unless specifically indicated otherwise. Although certain embodiments are disclosed herein, it is understood that these are provided by way of example only and should not be construed as limiting.

Those skilled in the art will therefore understand that various changes in form and detail may be made to the embodiments disclosed herein without departing from the scope of the following claims.

What is claimed is:

1. A water-mask material, comprising:
hydrogen ion water in the form of H⁺(H₂O)₂, H⁺(H₂O)₃, H⁺(H₂O)₄, H⁺(H₂O)₅, and/or H⁺(H₂O)₆;
wherein O is oxygen, H is hydrogen, and H⁺ is a positively charged hydrogen ion, the hydrogen ion of the hydrogen ion water thereby surrounded by between 2 and 6 water molecules.

2. The water-mask material of claim 1, wherein the hydrogen ion water includes between 10¹⁸ and 10²¹ hydrogen ions/liter.

3. The water-mask material of claim 1, further comprising 0.01-1% sodium chloride by weight.

4. The water-mask material of claim 1, further comprising 0.01-20% glycerol by weight.

5. The water-mask material of claim 1, further comprising 0.01-20% 1, 3-butanediol by weight.

6. The water-mask material of claim 1, further comprising:
00.01-1% sodium chloride by weight;
0.01-20% glycerol by weight; and
0.01-20% 1, 3-butanediol by weight.

7. The water-mask material of claim 1, further comprising:
0.5-1% sodium chloride by weight;
5-20% glycerol by weight; and
5-20% 1, 3-butanediol by weight.

8. The water-mask material of claim 1, wherein the cosmetic mask is provided as a sterilized, packaged liquid.

9. The water-mask material of claim 1, wherein the cosmetic mask includes hydrogen ions in a concentration of 10⁻² mol/L to 10⁻⁶ mol/L.

10. A method of making a water mask, comprising:
placing pure water in an electric field to form hydrogen ion water in the form of H⁺(H₂O)₂, H⁺(H₂O)₃, H⁺(H₂O)₄, H⁺(H₂O)₅, and/or H⁺(H₂O)₆;
packaging the hydrogen ion water; and
sterilizing the hydrogen ion water,
wherein O is oxygen, H is hydrogen, and H⁺ is a positively charged hydrogen ion, the hydrogen ion of the hydrogen ion water thereby surrounded by between 2 and 6 water molecules.

11. The method of claim 10, wherein sterilizing the hydrogen ion water includes exposing the hydrogen ion water to radiation.

12. The method of claim 10, wherein packaging the hydrogen ion water includes adding the following materials to the hydrogen ion water:
00.01-1% sodium chloride by weight;
0.01-20% glycerol by weight; and
0.01-20% 1, 3-butanediol by weight.

13. The method of claim 10, wherein packaging the hydrogen ion water includes adding the following materials to the hydrogen ion water:
0.5-1% sodium chloride by weight;
5-20% glycerol by weight; and
5-20% 1, 3-butanediol by weight.
